(19) United States
(12) Patent Application Publication
(10) Pub. No.: US 2021/0100451 A1
(43) Pub. Date: Apr. 8, 2021

(54) SYSTEMS AND METHODS OF REMOTE EXTRACTION OF SKELETAL INFORMATION USING MILLIMETER WAVE RADAR

(71) Applicants: Siyang Cao, Tucson, AZ (US); Arindam Sengupta, Tucson, AZ (US)

(72) Inventors: Siyang Cao, Tucson, AZ (US); Arindam Sengupta, Tucson, AZ (US)

(21) Appl. No.: 17/065,476

(22) Filed: Oct. 7, 2020

Related U.S. Application Data
(60) Provisional application No. 62/911,886, filed on Oct. 7, 2019.

(51) Int. Cl. A61B 5/00; A61B 5/11; G06N 3/02

(52) U.S. Cl. CPC A61B 5/0015; A61B 5/0004; A61B 5/72; G06N 3/02; A61B 5/7475; A61B 5/1116; A61B 5/45

(57) ABSTRACT

The systems and methods described herein provide a skeletal pose detection system using a mmWave radar sensor array, signal processing circuitry to generate a point cloud output using the mmWave sensor output signal, data processing circuitry to generate one or more point cloud intensity outputs using the point clout output, and AI circuitry to identify skeletal joints for each of one or more objects detected by the sensor array. The system may further include skeletal pose analysis circuitry to determine whether the skeletal joint arrangement associated with each of the one or more objects detected by the sensor array represent an arrangement indicative of a potential medical issue or other issue requiring attention and/or intervention.

### TECHNICAL FIELD

The present disclosure relates to detection of a skeletal pose using millimeter wave radar transceivers.

### BACKGROUND

Continued direct monitoring for patient motions in hospitals is largely deficient due to limited manpower and nursing resources. This can lead to windows of “unsupervised care” which increases health care utilization and decreases quality of life, especially for patients with bone cancer, Parkinson's disease, or mental disorders. Generally, sensors for motion monitoring can be classified into two categories: wearable and remote sensors. Wearable sensors require patients to carry devices attached to their body, such as headbands, smartwatches, sociometric badges, etc., to collect biometric and behavioral feedback. However, according to a public survey, the initial promise set out by wearable sensors would not be translated into a long-term commitment for several reasons: (i) non-pressing need, (ii) easy to misplace, (iii) unattractive aesthetics, (iv) uncomfortable during prolonged use, and (v) short-lived battery life, to name a few.

One type of remote sensor is the vision-based sensor, such as cameras, depth sensors, and the Microsoft Kinect (a combination of vision sensors), which can provide support for monitoring patient activity. However, vision-based sensors are ineffective under poor lighting conditions or during night, or when the sensor is occluded by dirt on its lens surface or smoke/steam in the monitoring area. Furthermore, there is an increased concern on patients' privacy, which greatly limits the wide use of vision-based sensors for medical/health care.

Another type of remote sensor is the radio frequency (RF) based sensor, such as Wi-Fi.

They use their own radio signals to illuminate the target, which allows RF sensors to be operationally robust, with no hindrance to their performance at night or even during occlusion. The RF signals from Wi-Fi can be used to measure the movement of patients and determine specific motions of a single person in the scenario with reasonable accuracy. However, primarily designated for data communication purposes, Wi-Fi does not have a wide-bandwidth signal and range measurement for distinguishing targets. That is, using Wi-Fi, we cannot get a contour/skeleton of the patient, and further distinguish a variety of specific motion behaviors.

### BRIEF DESCRIPTION OF THE DRAWINGS

Features and advantages of various embodiments of the claimed subject matter will become apparent as the following Detailed Description proceeds, and upon reference to the Drawings, wherein like numerals designate like parts, and in which:

FIG. 1 is a simplified block diagram of an illustrative skeletal pose detection system that includes a plurality of mmWave sensors, signal conditioning circuitry, data conditioning circuitry, and artificial intelligence (AI) circuitry, in accordance with at least one embodiment described herein;

FIG. 2A is a schematic diagram of an illustrative mmWave sensor array that includes a first single-plane mmWave radar array arranged to resolve one or more objects along an azimuth plane and a second single-plane mmWave radar array arranged to resolve one or more objects along an elevation plane, in accordance with at least one embodiment described herein;

FIG. 2B is a schematic diagram of an illustrative mmWave sensor array that includes a multi-plane mmWave radar array arranged to resolve one or more objects along both an azimuth plane and an elevation plane, in accordance with at least one embodiment described herein;

FIG. 3 is a block diagram of an illustrative signal conditioning circuitry, in accordance with at least one embodiment described herein;

FIG. 4A is a representation of an illustrative point cloud depicted in a three-dimensional cartesian (x, y, z) coordinate space generated by the data conditioning circuitry, in accordance with at least one embodiment described herein;

FIG. 4B is a representation of an illustrative three-dimensional heat map depicted in a three-dimensional cartesian (x, y, z) coordinate space as generated by the data conditioning circuitry, in accordance with at least one embodiment described herein;

FIG. 4C is a representation of an illustrative first projection of the three-dimensional heat map on a depth-azimuth (X, Y) plane and an illustrative second projection of the three-dimensional heat map on a depth-elevation (X, Z) plane, in accordance with at least one embodiment described herein;

FIG. 5 is a representation of an illustrative AI circuitry that includes first convolutional neural network (CNN) circuitry to receive the first N×N×3 image generated by the data conditioning circuitry and second CNN circuitry to receive the second N×N×3 image generated by the data conditioning circuitry, concatenation circuitry, flattening circuitry, and multilayer perceptron circuitry to provide an output signal containing information and/or data associated with a plurality of skeletal joints included in the one or more objects detected by the sensor array, in accordance with at least one embodiment described herein;

FIG. 6 is a high-level flow diagram of an illustrative method of determining the skeletal pose of one or more objects using a mmWave radar transceiver, in accordance with at least one embodiment described herein; and

FIG. 7 is a high-level flow diagram of an illustrative method of identifying a skeletal pose associated with each of the one or more objects detected by the sensor array, in accordance with at least one embodiment described herein.

Although the following Detailed Description will proceed with reference being made to illustrative embodiments, many alternatives, modifications and variations thereof will be apparent to those skilled in the art.

### DETAILED DESCRIPTION

The systems and methods disclosed herein are able to detect a skeletal pose (standing, sitting, prone, etc.) using data acquired using a multidimensional millimeter wave radar transceiver. The systems and methods disclosed herein are beneficially able to detect, predict, and report critical patients' behaviors (such as falling, seizure, etc.) to appropriate personnel during periods of “unsupervised care.” The systems and methods disclosed herein make use of a deployable mmWave radar-based motion monitoring system. Such systems and methods may be used to discern the skeletal movement of animate objects (humans, animals, etc.), and may be beneficially employed in health care applications, first responder situations, and in numerous military applications. The mmWave systems disclosed herein advantageously operate in low-light situations, such as night, and by providing an image containing only a skeletal depiction of an individual does not raise privacy concerns present with traditional vision-based sensors, such as cameras and infrared cameras. The mmWave systems disclosed herein feature relatively low cost (e.g., approx. $500), relatively small footprint (e.g., approx. 10 cm by 1 0cm by 8 cm), relatively low weight (e.g., approx. 300 grams). Beneficially, the systems and methods disclosed herein provide privacy protection, limited exposure to high-energy electromagnetic radiation, and are easily deployable.

Typically, radar is designed to measure and distinguish targets in range, angle, and velocity. Since 2017, a new technology—Radio Frequency Integrated Circuit (RFIC) for millimeter wave (mmWave) radar—has become available at a much lower cost and reduced physical size. The systems and methods disclosed herein beneficially make use of emergent mmWave radar technology to distinguish individuals and provide a real-time or near-real time output that includes information indicative of a skeletal pose of the individual.

The systems and methods disclosed herein include a signal processing circuitry to receive data from the mmWave transceivers and generate a point-cloud output signal that contains multi-dimensional data associated with one or more objects detected by the mmWave transceivers. The systems and methods disclosed herein further include data conditioning circuitry to generate a reduced output data set that includes at least location and reflection intensity associated with each of the points included in each of the detected objects. The systems and methods disclosed herein further include artificial intelligence circuitry to generate an output that includes information and/or data representative of a skeletal pose associated with each of the detected objects.

A system to detect the position of a plurality of skeletal joints is provided. The system may include: signal processing circuitry to: receive at least one millimeter wave (mmWave) radar input signal that includes information associated with one or more objects; and generate a point cloud output signal containing multi-dimensional data associated with the one or more objects. The system may further include data conditioning circuitry coupled to the signal processing circuitry, the data conditioning circuitry to: receive the point cloud output signal generated by the signal processing circuitry; and generate a data conditioning output signal that includes data representative of point cloud intensity information using at least a portion of the multi-dimensional data the received signal processing circuitry output signal. The system may additionally include artificial intelligence (AI) circuitry to: receive the data conditioning circuitry output signal; and generate, using the data representative of point cloud intensity information, at least one output signal that includes information associated with a location of each of a plurality of skeletal joints for each of at least a portion of the one or more objects.

A method to detect a plurality of skeletal joints is provided. The method may include receiving, by signal processing circuitry, at least one millimeter wave (mmWave) radar input signal that includes information associated with each of one or more objects. The method may further include generating, by the signal processing circuitry, a point cloud output signal containing multi-dimensional data corresponding to the one or more objects. The method may additionally include determining, by data conditioning circuitry coupled to the signal processing circuitry, point cloud intensity information using at least a portion of the multi-dimensional data corresponding to the one or more objects. The method may further include determining, by artificial intelligence circuity coupled to the data conditioning circuitry, a location of each of a plurality of skeletal joints for each of the one or more objects using point cloud density information.

A non-transitory computer readable medium is provided. The non-transitory computer readable medium may include instructions that, when executed by processor circuitry, cause the processor circuitry to: cause signal processing circuitry to generate a point cloud output signal containing multi-dimensional data corresponding to one or more objects detected by at least one communicably coupled millimeter wave (mmWave) radar transceiver; cause data conditioning circuitry coupled to the signal processing circuitry to determine point cloud intensity information using at least a portion of the multi-dimensional data corresponding to the one or more objects; cause the data conditioning circuitry to communicate the determined point cloud intensity information to communicably coupled artificial intelligence (AI) circuitry; and cause the AI circuitry to determine a location of each of a plurality of skeletal joints for each of the one or more objects.

As used herein, the terms “millimeter wave” and “mmWave” refer to systems and devices operating the 30 gigahertz (GHz) to 300 GHz electromagnetic spectral band.

As used herein, the term “artificial intelligence” and the term “artificial intelligence circuitry” refer to any system, device, circuitry, optical device, quantum computing device, or any combination thereof capable of: receiving one or more inputs at an input layer; passing all or a portion of the received input data and/or generated intermediate data either unidirectionally or bidirectionally through a series of nodes weighted using one or more training data sets; and generating output data at an output layer. The terms “artificial intelligence” and the term “artificial intelligence circuitry” may thus refer to any currently available and/or future developed neural network topology, any currently available and/or future developed multilayer perceptron topology, or combinations thereof.

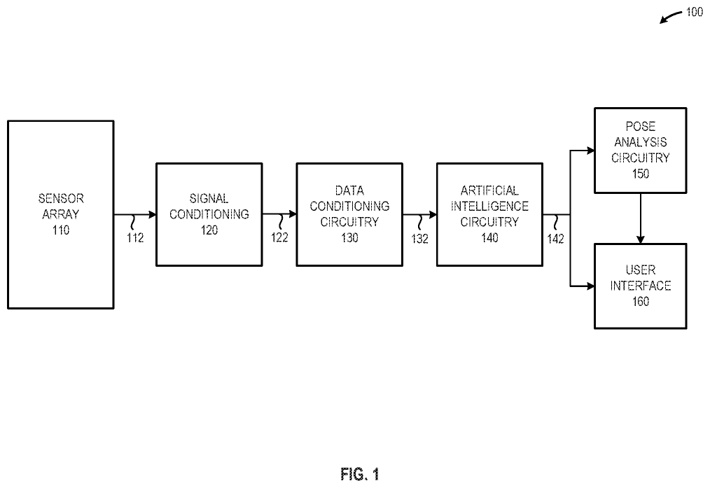

FIG. 1 is a simplified block diagram of an illustrative skeletal pose detection system 100 that includes a plurality of mmWave sensors 110, signal conditioning circuitry 120, data conditioning circuitry 130, and artificial intelligence (AI) circuitry 140, in accordance with at least one embodiment described herein. As depicted in FIG. 1, the skeletal pose output 142 from the AI circuitry 140 may be forwarded to analysis circuitry 150 to determine whether one or more skeletal poses indicate a potential medical situation (e.g., sitting proximate floor, laying proximate floor, unnatural pose, and similar). The skeletal pose detection system 100 may include a user interface 160 to display skeletal pose data and/or alert or warning data upon detection of a potential medical situation.

The sensor array 110 may include any number and/or combination of any currently available and/or future developed RF sensing devices capable of detecting the presence of objects, such as humans, in an area defined by the field-of-view of the sensing devices. In at least some embodiments, the sensor array may include any number of millimeter wave (mmWave) radar transceivers capable of detecting the presence of objects, such as humans, within the field of view of the mmWave transceivers. The sensor array 110 may be configured to detect the location of each detection point on one or more objects in a three dimensional space, for example in an orthogonal x, y, z cartesian coordinate system. The sensor array 110 may also determine a reflectivity or reflection intensity value for each detection point on each of the one or more objects in the three dimensional space. In some embodiments, the sensor array 110 may include one or more sensors capable of detecting and locating detected points on the one or more objects within a three dimensional space. In other embodiments, the sensor array 110 may include a plurality of sensors, each capable of detecting an locating point on the one or more objects within a planar or two-dimensional space. Such two-dimensional sensors may be positioned (e.g., orthogonally) so as to provide location information for each point on the one or more objects in a three-dimensional space. One or more signals 112 communicate the data collected by the sensor array 110 to the signal conditioning circuitry 120.

The signal conditioning circuitry 120 may include circuitry having any number and/or combination of currently available and/or future developed circuitry that includes electronic components, optical components, semiconductor devices, and/or logic elements capable of generating multi-dimensional point cloud output 122 that includes point location and intensity information based on signal(s) provided by the sensor array 110. In at least some embodiments, the signal conditioning circuitry 120 may include circuitry configured to provide a range, radial speed, angle, and point reflectivity strength for each detected point on each of the one or more objects detected by the sensor array 110 in the three dimensional space.

The data conditioning circuitry 130 may include circuitry having any number and/or combination of currently available and/or future developed circuitry that includes electronic components, optical components, semiconductor devices, and/or logic elements capable of generating a reduced data set output 132 that includes the point location and reflectivity information included in the multi-dimensional point cloud produced by the signal conditioning circuitry 120. In embodiments, the reduced data set may include a plurality of two-dimensional data sets, each of which includes location information for each point on the one or more objects detected by the sensor array 110 in the three dimensional space. In embodiments, the reduced data set is provided as an input to the AI circuitry 140.

The AI circuitry 140 may include any number and/or combination of currently available and/or future developed circuitry that includes electronic components, optical components, semiconductor devices, and/or logic elements capable of generating a skeletal pose output for each of some or all of the one or more objects detected by the sensor array 110. In embodiments, the AI circuitry 140 may include any number and/or combination of neural networks, multilayer perceptron networks, and hybrid networks. In embodiments, the AI circuitry 140 maps each of the points on the one or more objects detected by the sensor array 110 to a distinct skeletal joint of the human body in a three-dimensional space. The AI circuitry 140 generates an output 142 that includes the skeletal pose for each of at least some of the one or more objects detected by the sensor array 110.

The pose analysis circuitry 150 may include any number and/or combination of currently available and/or future developed circuitry that includes electronic components, optical components, semiconductor devices, and/or logic elements capable of evaluating the skeletal pose of each of the one or more objects detected by the sensor array 110 to determine whether the skeletal pose of the object is indicative of a possible medical or other emergency situation. For example, the pose analysis circuitry 150 may include circuitry configured to identify whether the skeletal pose is indicative of a kneeling or prone individual.

The user interface 160 may include any number and/or combination of currently available and/or future developed systems or devices capable of providing a human perceptible output (e.g., an audio output device, a video output device, a tactile output device, or combinations thereof). In embodiments, the user interface 160 may provide an output representative of the skeletal pose of each of the one or more objects detected by the sensor array 110. In embodiments, the user interface 160 may provide an output representative of an alert or similar notification upon detecting a skeletal pose indicative of a potential medical or alert condition.

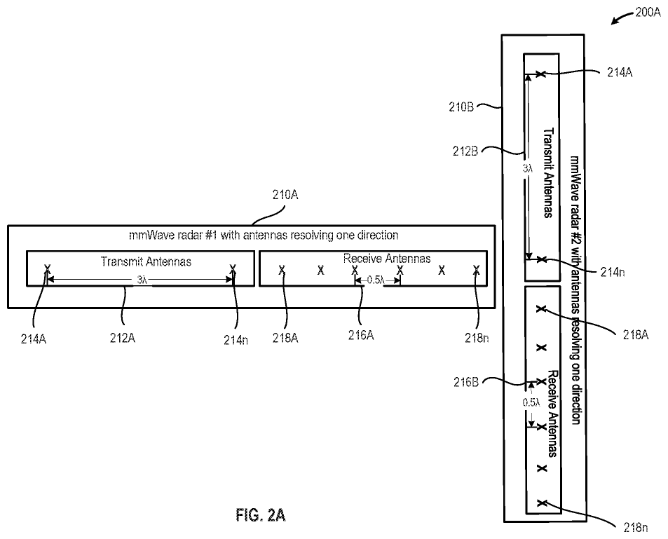

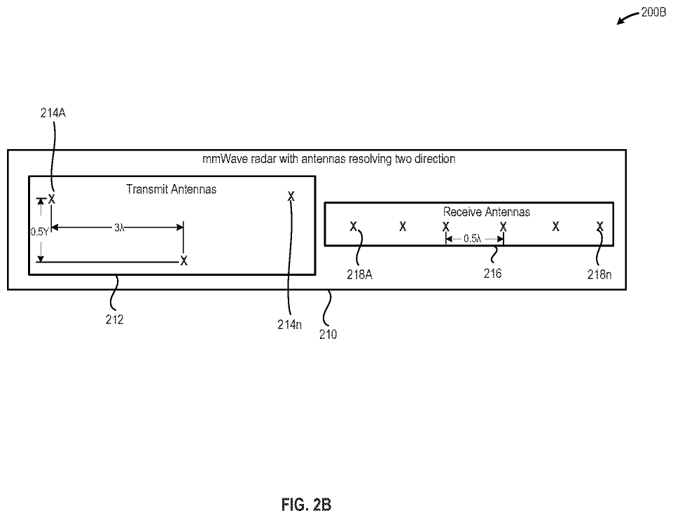

FIG. 2A is a schematic diagram of an illustrative mmWave sensor array 200A that includes a first single-plane mmWave radar array 210A arranged to resolve one or more objects along an azimuth plane and a second single-plane mmWave radar array 210B arranged to resolve one or more objects along an elevation plane, in accordance with at least one embodiment described herein. FIG. 2B is a schematic diagram of an illustrative mmWave sensor array 200B that includes a multi-plane mmWave radar array arranged to resolve one or more objects along both an azimuth plane and an elevation plane, in accordance with at least one embodiment described herein.

Turning first to FIG. 2A, the first single-plane mmWave radar array 210A includes a mmWave transmission array 212A that includes a plurality of transmission antennas 214A-214n (collectively, “transmission antennas 214”). In embodiments the transmission antennas 214 may be spaced apart a defined distance based on the wavelength of the transmitted mmWave signal. For example, the transmission antennas 214 may be spaced at intervals of: 1 wavelength or less; 2 wavelengths or less; 3 wavelengths or less; 5 wavelengths or less; 10 wavelengths or less; or 20 wavelengths or less. The first single-plane mmWave radar array 210A includes a mmWave receiver array 216A that includes a plurality of receiver antennas 218A-218n (collectively, “receiver antennas 218”). In embodiments the receiver antennas 218 may be spaced apart a defined distance based on the wavelength of the transmitted mmWave signal. For example, the receiver antennas 218 may be spaced at intervals of: 0.1 wavelength or less; 0.25 wavelength or less; 0.50 wavelength or less; 1 wavelength or less; 2 wavelengths or less; 3 wavelengths or less; 5 wavelengths or less; or 10 wavelengths or less. The first single-plane mmWave radar array 210A may resolve the one or more objects in an angle across an azimuthal plane.

The second single-plane mmWave radar array 210B includes a mmWave transmission array 212B that includes a plurality of transmission antennas 214A-214n (collectively, “transmission antennas 214”). In embodiments the transmission antennas 214 may be spaced apart a defined distance based on the wavelength of the transmitted mmWave signal. For example, the transmission antennas 214 may be spaced at intervals of: 1 wavelength or less; 2 wavelengths or less; 3 wavelengths or less; 5 wavelengths or less; 10 wavelengths or less; or 20 wavelengths or less. The number of the transmit antennas may be more than 2, and the number of the receive antenna may also be more than 6. The second single-plane mmWave radar array 210B includes a mmWave receiver array 216B that includes a plurality of receiver antennas 218A-218n (collectively, “receiver antennas 218”). In embodiments the receiver antennas 218 may be spaced apart a defined distance based on the wavelength of the transmitted mmWave signal. For example, the receiver antennas 218 may be spaced at intervals of: 0.1 wavelength or less; 0.25 wavelength or less; 0.50 wavelength or less; 1 wavelength or less; 2 wavelengths or less; 3 wavelengths or less; 5 wavelengths or less; or 10 wavelengths or less. The number of the transmit antennas may be more than 2, and the number of the receive antenna may also be more than 6. The second single-plane mmWave radar array 210B may resolve the one or more objects in an angle across an elevational plane. Together, the first single-plane mmWave radar array 210A and the second single-plane mmWave radar array 210B may resolve and/or detect one or more objects in three dimensional space.

Turning next to FIG. 2B, the multi-plane mmWave radar array 210 includes a mmWave transmission array 212 that includes a plurality of transmission antennas 214A-214n (collectively, “transmission antennas 214”) arranged along a first axis and a second axis orthogonal to the first axis. In embodiments the transmission antennas 214 may be spaced apart along the first axis a defined distance based on the wavelength of the transmitted mmWave signal. For example, the transmission antennas 214 may be spaced along the first axis at intervals of: 1 wavelength or less; 2 wavelengths or less; 3 wavelengths or less; 5 wavelengths or less; 10 wavelengths or less; or 20 wavelengths or less. In embodiments the transmission antennas 214 may be spaced apart along the second axis a defined distance based on the wavelength of the transmitted mmWave signal. For example, the transmission antennas 214 may be spaced along the second axis at intervals of: 0.1 wavelength or less; 0.25 wavelength or less; 0.50 wavelength or less; 1 wavelength or less; 2 wavelengths or less; 3 wavelengths or less; 5 wavelengths or less; or 10 wavelengths or less.

The multi-plane mmWave radar array 210 includes a mmWave receiver array 216 that includes a plurality of receiver antennas 218A-218n (collectively, “receiver antennas 218”). In embodiments the receiver antennas 218 may be spaced apart a defined distance based on the wavelength of the transmitted mmWave signal. For example, the receiver antennas 218 may be spaced at intervals of: 00.1 wavelength or less; 0.25 wavelength or less; 0.50 wavelength or less; 1 wavelength or less; 2 wavelengths or less; 3 wavelengths or less; 5 wavelengths or less; or 10 wavelengths or less. The multi-plane mmWave radar array 210 may resolve the one or more objects in both: an angle across an azimuthal plane and an angle across an elevational plane. The number of transmit antennas in multi-plane mmWave radar array 210 can be more than 3, and the number of receive antennas in multi-plane mmWave radar array 210 can be more than 6.

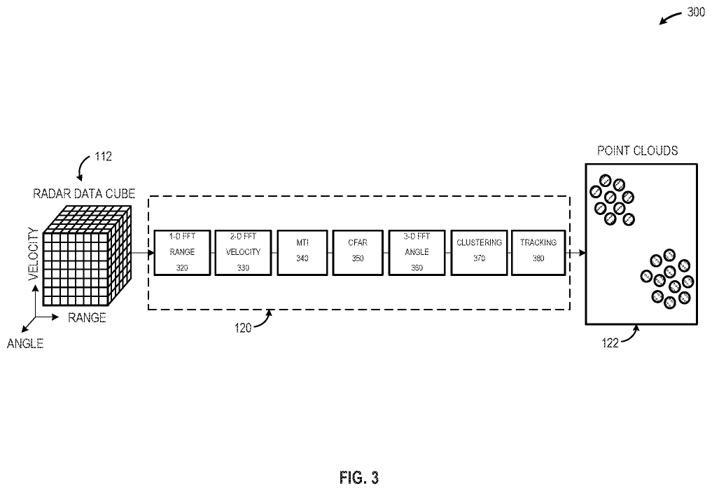

FIG. 3 is a block diagram of an illustrative signal conditioning circuitry 120, in accordance with at least one embodiment described herein. As depicted in FIG. 3, in at least some embodiments, the signal conditioning circuitry 120 may include Fast Fourier Transform (FFT) circuitry 320 to transform the data 112 received from the sensor array 110 in a first (e.g., range) dimension. The signal conditioning circuitry 120 may include Fast Fourier Transform (FFT) circuitry 330 to transform the data 112 received from the sensor array 110 in a second (e.g., velocity) dimension. The signal conditioning circuitry 120 may include MTI circuitry 340 and CFAR circuitry 350. The signal conditioning circuitry 120 may include Fast Fourier Transform (FFT) circuitry 360 to transform the data 112 received from the sensor array 110 in a third (e.g., angle) dimension. The signal conditioning circuitry 120 also includes clustering circuitry 370 and tracking circuitry 380 so that points corresponding to different objects may be clustered. The output signal 122 generated by the data conditioning circuitry 120 includes data representative of a point cloud in which each point in each of the one or more objects included in the point cloud includes data representative of: a) the range of the point; b) the radial speed of the point; c) the angle of the point; and d) the target reflectivity strength.

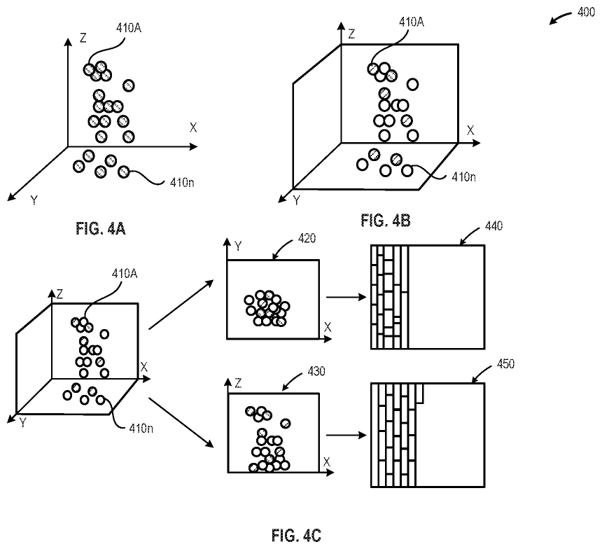

FIG. 4A is a representation of an illustrative point cloud 400A depicted in a three-dimensional x, y, z coordinate space generated by the data conditioning circuitry 130, in accordance with at least one embodiment described herein. In addition to coordinates identifying a location in three-dimensional space, each point 410A-410n also includes a value representative of the reflectance or absorbance of the respective point to the mmWave signal generated by the mmWave sensor array 110. For example, an RGB pixel value may be assigned to each point included in the point cloud to create a three-dimensional heat map.

FIG. 4B is a representation of an illustrative three-dimensional heat map 400B depicted in a three-dimensional x, y, z coordinate space as generated by the data conditioning circuitry 130, in accordance with at least one embodiment described herein. As depicted in the three-dimensional heat map 400B, each of the points 410A-410n has been assigned an RGB pixel value corresponding to the reflectance of the respective point, in accordance with at least one embodiment described herein. In embodiments, such a three-dimensional heat map 400B may be used as an input to the AI circuitry 140. However, the data dimension of the three-dimensional heat map 400B may be too great for direct input to the AI circuitry 140.

FIG. 4C is a representation of an illustrative first projection of the three-dimensional heat map 400B on a depth-azimuth (X, Y) plane 420 and an illustrative second projection of the three-dimensional heat map 400B on a depth-elevation (X, Z) plane 430, in accordance with at least one embodiment described herein. Each pixel may be assigned an RGB value (I) that represents the reflectivity of the point on the one or more objects corresponding to the indicated pixel. In embodiments, pixels that do not correspond to a point on the one or more objects may be assigned a (0,0,0) value in the RGB channels. The first projection of the three-dimensional heat map 400B on a depth-azimuth (X, Y) plane 420 produces a first N×N×3 image 440 with (X,Y,I) as the RGB channel The second projection of the three-dimensional heat map 400B on a depth-elevation (X, Z) plane 430 produces a second N×N×3 image 450 with (X,Z,I) as the RGB channel. If the actual number of points detected is fewer than N², the remaining pixels corresponding to no detection would be assigned with a (0,0,0) in the RGB channels.

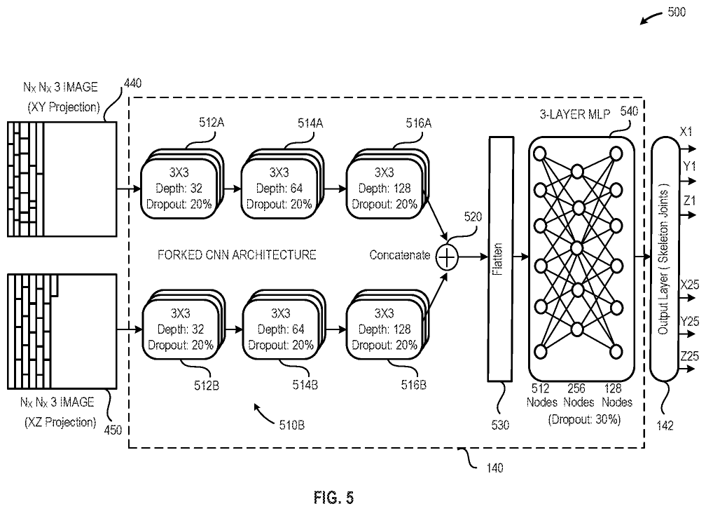

FIG. 5 is a representation of an illustrative AI circuitry 140 that includes first convolutional neural network (CNN) circuitry 510A to receive the first N×N×3 image 440 generated by the data conditioning circuitry 130 and second CNN circuitry 510B to receive the second N×N×3 image 450 generated by the data conditioning circuitry 130, concatenation circuitry 520, flattening circuitry 530, and a multilayer perceptron 540 to provide an output signal containing information and/or data associated with a plurality of skeletal joints included in the one or more objects detected by the sensor array 110, in accordance with at least one embodiment described herein. Although FIG. 5 depicts a plurality of convolutional neural networks, one may readily appreciate that any number and/or combination of currently available and/or future developed neural network circuits may be similarly employed as described herein. In some embodiments, CNN circuitry 510A and/or 510B may comply or be compatible with standardized CNN protocols and toolkits, which may include, for example, Caffe, Deeplearning4j, Dilb, TensorFlow, Theano, Torch, etc., and/or other standardized CNN protocols and toolkits and/or custom CNN protocols and toolkits and/or after-developed CNN protocols and toolkits.

In embodiments, the first CNN circuitry 510A includes a first CNN circuit 512A having a depth of 32 bits; a second CNN circuit 514A having a depth of 64 bits; and, a third CNN circuit 516A having a depth of 128 bits. In other embodiments, CNN circuits having different bit depths may be substituted in the first CNN circuitry 510A. The output from the first CNN circuitry 510A may have output dimensions of N×N×128. In embodiments, the second CNN circuitry 510B may include a first CNN circuit 512B having a depth of 32 bits; a second CNN circuit 514B having a depth of 64 bits; and, a third CNN circuit 516B having a depth of 128 bits. The output from the second CNN circuitry 510B may have output dimensions of N×N×128. In other embodiments, CNN circuits having different bit depths may be substituted in the second

CNN circuitry 510B. In embodiments, the filter size can be set at 3×3 with a single-pixel stride and same padding. The nodes can be activated using Leaky Relu (alpha=0.3) with a 20% dropout to avoid overfitting. These numbers can also be adjusted according to the specific application.

Concatenation circuitry 520 concatenates the outputs from the first CNN circuitry 510A and the second CNN circuitry 510B to form a N×N×256 tensor. Flattening circuitry 530 then flattens the N×N×256 tensor. The flattened N×N×256 tensor is provided to multilayer perceptron (MLP) circuitry 540. In embodiments, the MLP circuitry 540 beneficially accommodates the non-linear modeling of the input signal(s) received from the sensor array 110 with respective ones of each of a plurality of skeletal joints on each of the one or more objects detected by the sensor array 110. In at least some embodiments, the MLP circuitry 540 may have three layers, a 512 node layer, a 256 node layer, and a 128 node layer. In at least some embodiments, the MLP circuitry 540 may have a 30% dropout and Leaky Relu (alpha=0.3) activation function.

The system 100 accurately maps the radar reflection points received from the sensor array 110 to a plurality of (e.g., 25) “n” distinct skeletal joints of the human body in 3-D space. Therefore, the output layer of the MLP circuitry 540 consists of “3*n” nodes (e.g., 75 nodes) that corresponding to the (X,Y,Z) coordinates of each of the “n” joints. The output layer has a linear activation function and is fully-connected to the final layer of the MLP circuitry 540. The model is trained with the objective to minimize the mean-squared-error (MSE) of the predicted location of the joints with the measured ground truth. The model is trained using gradient descent using the Adam optimizer, that uses a variable learning rate depending on the rate of change of the gradient over iterations.

The added advantage the systems and methods disclosed herein is that this approach would not only work with radar systems that have both azimuth and elevation channels (FIG. 2), but can also be extended to radar modules that only have antenna elements in one axis (FIG. 1). In the latter case, two radars can then be used, with one capturing XY data and the other rotated at 90° to capture XZ data. This way N×N×3 images can be directly generated with no projection operation required as each radar detects the reflected points in the respective single plane. The proposed approach also eliminates the need for data association or complex construction of 4D CNNs. Finally, by incorporating the reflection power levels, we provide the CNN with an additional feature to aid the learning process and distinguish between the reflections from a larger RCS of the body (e.g., torso) from a smaller RCS (e.g., elbow).

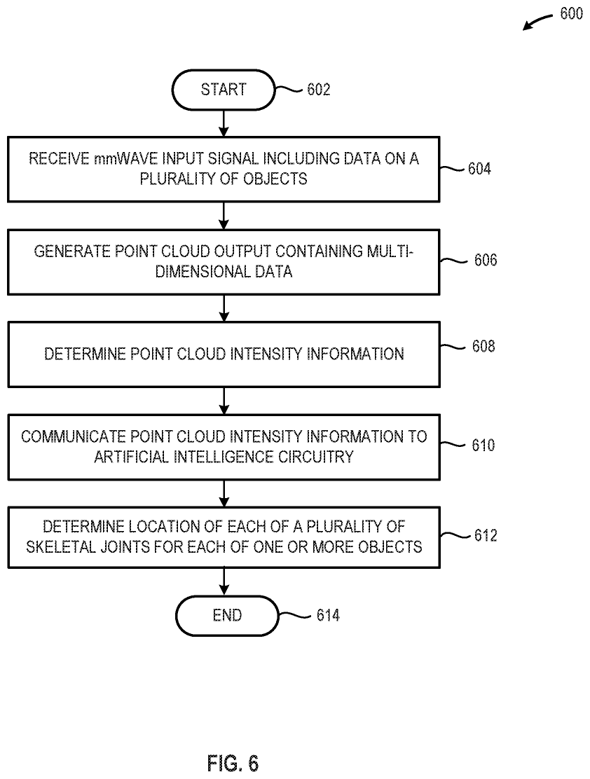

FIG. 6 is a high-level flow diagram of an illustrative method 600 of determining the skeletal pose of one or more objects using a mmWave radar transceiver, in accordance with at least one embodiment described herein. The system may include a sensor array 110 that includes any number of mmWave radar transceivers to generate a data output that includes point data associated with each of one or more objects disposed within the angular field of view of the sensor array 110. Signal processing circuitry 120 and data processing circuitry 130 organize the data received from the sensor array 110 into a format useful by the artificial intelligence circuitry 140 to identify skeletal joints associated with each of the one or more objects. The method 600 commences at 602.

At 604, the signal processing circuitry 120 receives from the sensor array 110 one or more signals conveying, carrying, transporting, or otherwise transferring information and/or data associated with one or more objects detected by the sensor array 110.

At 606, the signal processing circuitry 120 generates a point cloud output signal 122 that includes at least three-dimensional location information corresponding to each point on the one or more objects detected by the sensor array 110. In embodiments, the signal processing circuitry 120 may generate a point cloud output signal 122 that includes reflection intensity information associated with each point on the one or more objects detected by the sensor array 110. In some embodiments, the point location and intensity information associated with each point on the one or more objects detected by the sensor array 110 provides a four-dimensional array. The four-dimensional data generated by the signal processing circuitry 120 may, in some instances, be sufficiently voluminous to adversely affect the responsiveness of the AI circuitry 140.

At 608, the data processing circuitry 130 determines the point cloud intensity information in a format amenable to further processing by the AI circuitry 140. In embodiments, the data processing circuitry 130 formats the point cloud data received from the signal processing circuitry 120 into a plurality of two-dimensional heat maps 440, 450 that are communicated to the AI circuitry 140.

At 610, the data processing circuitry 130 communicates the plurality of two-dimensional heat maps 440, 450 to the AI circuitry 140.

At 612, the AI circuitry 140, using the plurality of two-dimensional heat maps 440, 450 to determine the location of a plurality of skeletal joints associated with each of the one or more objects detected by the sensor array 110. The method 600 concludes at 614.

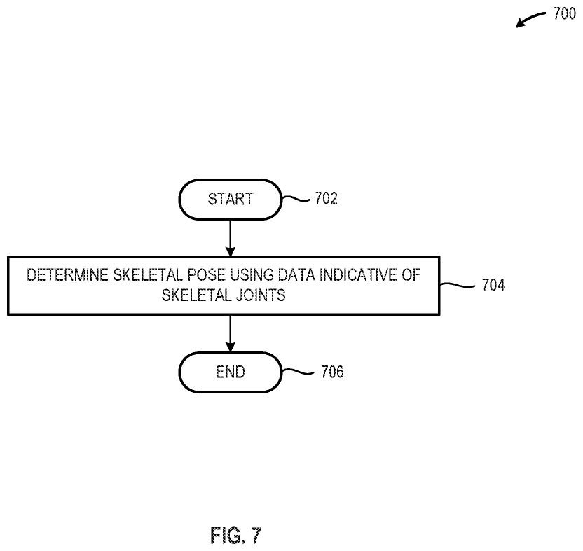

FIG. 7 is a high-level flow diagram of an illustrative method 700 of identifying a skeletal pose associated with each of the one or more objects detected by the sensor array 110, in accordance with at least one embodiment described herein. The method 700 may be used in conjunction with the method 600 described above. The method 700 commences at 702.

At 704, skeletal pose analysis circuitry 150 determines a skeletal pose associated with each of the one or more objects identified by the sensor array 110. In such embodiments, the skeletal pose analysis circuitry 150 uses at least a portion of the information and/or data included in the output signal 142 generated by the AI circuitry 140. The method 700 concludes at 706.

While FIGS. 6 and 7 illustrate vehicular force absorption system according to one or more embodiments, it is to be understood that not all of the operations depicted in FIGS. 6 and 7 may be necessary for other embodiments. Indeed, it is fully contemplated herein that in other embodiments of the present disclosure, the operations depicted in FIGS. 6 and 7, and/or other operations described herein, may be combined in a manner not specifically shown in any of the drawings, but still fully consistent with the present disclosure. Thus, claims directed to features and/or operations that are not exactly shown in one drawing are deemed within the scope and content of the present disclosure.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

The systems and methods described herein provide a skeletal pose detection system using a mmWave radar sensor array, signal processing circuitry to generate a point cloud output using the mmWave sensor output signal, data processing circuitry to generate one or more point cloud intensity outputs using the point clout output, and AI circuitry to identify skeletal joints for each of one or more objects detected by the sensor array. The system may further include skeletal pose analysis circuitry to determine whether the skeletal joint arrangement associated with each of the one or more objects detected by the sensor array represent an arrangement indicative of a potential medical issue or other issue requiring attention and/or intervention.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

What is claimed:

1. A system to detect the position of a plurality of skeletal joints, the system comprising:
signal processing circuitry to:
receive at least one millimeter wave (mmWave) radar input signal that includes information associated with one or more objects; and
generate a point cloud output signal containing multi-dimensional data associated with the one or more objects;
data conditioning circuitry coupled to the signal processing circuitry, the data conditioning circuitry to:
receive the point cloud output signal generated by the signal processing circuitry; and
generate a data conditioning output signal that includes data representative of point cloud intensity information using at least a portion of the multi-dimensional data the received signal processing circuitry output signal;
artificial intelligence (AI) circuitry to:
receive the data conditioning circuitry output signal; and
generate, using the data representative of point cloud intensity information, at least one output signal that includes information associated with a location of each of a plurality of skeletal joints for each of at least a portion of the one or more objects.

2. The system of claim 1, further comprising:
one or more mmWave radar transceivers to generate the at least one mmWave radar input signal that includes the data associated with each of the one or more objects detected within a respective field-of-view of each of the one or more mmWave transceivers.

3. The system of claim 2 wherein the one or more mmWave radar transceivers comprise a first mono-planar mmWave transceiver aligned along a first detection plane and a second mono-planar mmWave transceiver aligned along a second detection plane orthogonal to the first detection plane, the first mono-planar mmWave transceiver and the second mono-planar mmWave transceiver to generate the data associated with each of the one or more objects detected within the field-of-view of the first mono-planar mmWave transceiver and the second mono-planar mmWave transceiver

4. The system of claim 2 wherein the one or more mmWave radar transceivers comprise at least one multi-planar mmWave transceiver, the at least one multi-planar mmWave transceiver to provide the data associated with each of the one or more objects detected within the field-of-view of the at least one multi-planar mmWave transceiver.

5. The system of claim 1, further comprising:
at least one output device to display the at least one output signal that includes information associated with the location of each of the plurality of skeletal joints for each of at least a portion of the one or more objects included in the at least one mmWave radar input signal.

6. The system of claim 1, the convolutional neural network further comprising circuitry to detect a pose of each of the one or more objects included in the at least one mmWave radar input signal using the location of each of the plurality of skeletal joints for each respective one of the one or more objects included in the at least one mmWave radar input signal.

7. The system of claim 1 wherein the point cloud output signal generated by the signal processing circuitry comprises at least one of: object clustering data or tracking data.

8. The system of claim 1 wherein the multi-dimensional data includes, for each point on the one or more objects included in the at least one mmWave radar input signal:
radial velocity data;
angle data;
range data; and
reflection strength.

9. The system of claim 1 wherein the data conditioning output signal comprises a plurality of output signals including:
a first data conditioning output signal that includes depth-azimuth (XY) and reflection intensity data in the form of an N×N×3 image; and
a second data conditioning output signal that includes depth-elevation (XZ) and reflection intensity data.

10. The system of claim 9 wherein the CNN circuitry comprises:
first neural network circuitry to receive the first data conditioning output signal that includes depth-azimuth (XY) and reflection intensity data to provide a first N×N×128 output signal;
second neural network circuitry to receive the a second data conditioning output signal that includes depth-elevation (XZ) and reflection intensity data to provide a second N×N×128 output signal;
data concatenation circuitry to concatenate the first N×N×128 output signal with the second N×N×128 output signal to generate an N×N×256 output tensor;
flattening circuitry to flatten the N×N×256 output tensor; and
multilayer perceptron circuitry to generate the at least one output signal that includes information associated with the location of each of the plurality of skeletal joints for each of at least a portion of the one or more objects.

11. A method to detect a plurality of skeletal joints, comprising:
receiving, by signal processing circuitry, at least one millimeter wave (mmWave) radar input signal that includes information associated with each of one or more objects;
generating, by the signal processing circuitry, a point cloud output signal containing multi-dimensional data corresponding to the one or more objects;
determining, by data conditioning circuitry coupled to the signal processing circuitry, point cloud intensity information using at least a portion of the multi-dimensional data corresponding to the one or more objects; and
determining, by artificial intelligence circuity coupled to the data conditioning circuitry, a location of each of a plurality of skeletal joints for each of the one or more objects using point cloud density information.

12. The method of claim 11 wherein generating the point cloud output signal containing the multi-dimensional data corresponding to the one or more objects further comprises:
generating, by the signal processing circuitry, a four-dimensional point cloud output signal that includes, for each point in each of the one or more objects, data representative of:
a radial velocity of the respective point included in the detected object;
an angle of the respective point included in the detected object;
a range to the respective point included in the detected object;
a reflection strength of the respective point included in the detected object.

13. The method of claim 11, further comprising:
generating, by one or more mmWave radar transceivers, the at least one mmWave radar input signal that includes information associated with each of the one or more objects.

14. The method of claim 13 wherein generating the at least one mmWave radar input signal that includes information associated with each of the one or more objects further comprises:
generating, by a first mono-planar mmWave transceiver aligned along a first detection plane, a first mmWave radar signal that includes information associated with the one or more objects; and
generating, by a second mono-planar mmWave transceiver aligned along a second detection plane orthogonal to the first detection plane, a second mmWave radar signal that includes information associated with the one or more objects.

15. The method of claim 13 wherein generating the at least one mmWave radar input signal that includes information associated with each of the one or more objects further comprises:
generating, by at least one multi-planar mmWave transceiver, the information associated with each of the one or more objects.

16. The method of claim 11, further comprising:
communicating, to a communicably coupled user interface device, a signal that includes the location of each of a plurality of skeletal joints for each of the one or more objects.

17. The method of claim 16, further comprising:
detecting, by the artificial intelligence circuitry, a pose of each of the one or more objects using the location of each of the plurality of skeletal joints for each respective one of the one or more objects.

18. The method of claim 11 wherein determining point cloud intensity information using at least a portion of the multi-dimensional data corresponding to the one or more objects further comprises:
generating, by the data conditioning circuitry, a first data conditioning output signal that includes depth-azimuth (XY) and reflection intensity data in the form of an N×N×3 image; and
generating, by the data conditioning circuitry, a second data conditioning output signal that includes depth-elevation (XZ) and reflection intensity data.

19. The method of claim 18, wherein determining, by artificial intelligence circuity coupled to the data conditioning circuitry, a location of each of a plurality of skeletal joints for each of the one or more objects further comprises:
generating, by first neural network circuitry, a first N×N×128 output signal using the first data conditioning output signal that includes depth-azimuth (XY) and reflection intensity data;
generating, by second neural network circuitry, a second N×N×128 output signal using the second data conditioning output signal that includes depth-elevation (XZ) and reflection intensity data;
generating, by the AI circuitry an N×N×256 output tensor by concatenating the first N×N×128 output signal with the second N×N×128 output signal;
flattening, by the AI circuitry, the N×N×256 output tensor; and
generating, by multilayer perceptron circuitry, the at least one output signal that includes the location of each of the plurality of skeletal joints for each of the one or more objects.

20. A non-transitory computer readable medium including instructions that, when executed by processor circuitry, cause the processor circuitry to:
cause signal processing circuitry to generate a point cloud output signal containing multi-dimensional data corresponding to one or more objects detected by at least one communicably coupled millimeter wave (mmWave) radar transceiver;
cause data conditioning circuitry coupled to the signal processing circuitry to determine point cloud intensity information using at least a portion of the multi-dimensional data corresponding to the one or more objects;
cause the data conditioning circuitry to communicate the determined point cloud intensity information to communicably coupled artificial intelligence (AI) circuitry; and
cause the AI circuitry to determine a location of each of a plurality of skeletal joints for each of the one or more objects.
